(19) United States
(12) Patent Application Publication
(10) Pub. No.: US 2024/0381822 A1
(43) Pub. Date: Nov. 21, 2024

(54) PLANT PART QUALITY

(71) Applicant: Valent BioSciences LLC, Libertyville, IL (US)

(72) Inventors: Peter D. Petracek, Grayslake, IL (US); Veria Ysabel Alvarado Chavez, Grayslake, IL (US); Steve McArtney, Antioch, IL (US); Hanwei Zhang, Port Barrington, IL (US)

(21) Appl. No.: 18/663,582

(22) Filed: May 14, 2024

Related U.S. Application Data
(60) Provisional application No. 63/502,182, filed on May 15, 2023.

(51) Int. Cl. A01H 3/04; A01G 7/06; A01N 37/42; A01P 21/00

(52) U.S. Cl. CPC A01H 3/04; A01G 7/06; A01N 37/42; A01P 21/00

(57) ABSTRACT

The present invention is directed to methods of improving post-harvest cold stress tolerance in a plant part comprising applying an effective amount of a mixture of abscisic acid and a jasmonate to the plant part. The present invention is further directed to methods of improving plant part quality in plant parts subjected to post-harvest chilling temperatures comprising applying an effective amount of a mixture of (S)-abscisic acid and a jasmonate to the plant part.

### FIELD OF THE INVENTION

The present invention is directed to methods of improving post-harvest cold stress tolerance in a plant part comprising applying an effective amount of a mixture of abscisic acid and a jasmonate to the plant part. The present invention is further directed to methods of improving plant part quality in plant parts subjected to post-harvest chilling temperatures comprising applying an effective amount of a mixture of (S)-abscisic acid and a jasmonate to the plant part.

### BACKGROUND OF THE INVENTION

(S)-abscisic acid (“S-ABA”) is an endogenous plant growth regulator with many roles in growth and development. For example, S-ABA inhibits seed germination by antagonizing gibberellins that stimulate the germination of seeds. S-ABA promotes stress tolerance and maintains growth under stress conditions (see Sharp R E et al. J Exp Bot, 2004 55:2343-2351).

Jasmonates are phytohormones derived from cyclic fatty acids and regulates plant defenses to pests and further regulates developmental processes. Methyl jasmonate has been well studied and has been further found to be involved in root growth, growth of reproductive organs and plant senescence.

Plant parts such as fruits such as avocado, pomegranates, bananas and mangoes, vegetables such as carrots, lettuce and broccoli and flowers are important crops in the US and around the world. There is an increasing emphasis on high quality plant parts. Growers are now challenged to produce crops with adequate color and optimal flavor as consumers have grown to expect high quality on a year-round basis.

Chilling of the plant parts can lead to low quality. Specifically, chilling can damage the many plant part qualities that are desired by the consumer such as texture, color, firmness, juiciness, aroma, and flavor. Chilling damage can also lower hedonic scores, which are scores given by consumer taste panel assessments. However, refrigeration is often used to impede ripening and retard fungal growth on plant parts so that time from harvest to store shelf and store shelf life can be extended as both over ripe plant parts and fungal growth are undesirable to the consumer.

Thus, there is a need in the art for a method of improving cold tolerance and improving quality of plant parts that are subjected to chilling temperatures so that higher quality plant parts may be available to the consumer.

### SUMMARY OF THE INVENTION

In one aspect, the present invention is directed to methods of improving post-harvest cold stress tolerance in a plant part comprising applying an effective amount of a mixture of (S)-abscisic acid (“S-ABA”) and a jasmonate to the plant part.

In another aspect, the present invention is further directed to methods of improving plant part quality in plant parts subjected to post-harvest chilling temperatures comprising applying an effective amount of a mixture of S-ABA and a jasmonate to the plant part.

### DETAILED DESCRIPTION OF THE INVENTION

Applicant unexpected discovered that a mixture of (S)-abscisic acid (“S-ABA”) and a jasmonate synergistically improved post-harvest cold stress tolerance in many plant parts. Further, the Applicant unexpectedly discovered that a mixture of S-ABA and a jasmonate improved plant part quality when the plant part is subjected to post-harvest chilling temperatures.

In one embodiment, the present invention is directed to methods of improving post-harvest cold stress tolerance in a plant part comprising applying an effective amount of a mixture of S-ABA and a jasmonate to the plant part.

In a preferred embodiment, applications of the mixture of the present invention occur from about 4 weeks prior to harvest to about 2 weeks after harvest. In a more preferred embodiment application occurs after harvest.

In another preferred embodiment, the plant part is subjected to chilling temperatures following application of the mixture of the present invention, preferably from about 0.5 to about 20 degrees Celsius and more preferably from about 2 to about 10 degrees Celsius.

In another preferred embodiment, the quality of the plant part is improved as compared to plant parts that did not receive an application of the mixture of the present prior to being subjected to chilling temperatures.

In another preferred embodiment, the mixture of the present invention may be applied at a weight ratio of rom about 1,000:1 to about 1:1000 S-ABA to the jasmonate, more preferably from about 500:1 to about 1:500 and even more preferably from about 100:1 to about 1:100, yet even more preferably from about 10:1 to about 1:10 and most preferably from about 3:1 to about 1:3.

In another preferred embodiment, S-ABA may be applied at a rate from about 1 to about 5,000 parts per million (“ppm”), more preferably from about 10 to about 1,000 ppm, even more preferably from about 50 to about 1,000 ppm and even more preferably at about 90, about 320 or about 870 ppm.

In another preferred embodiment, the jasmonate may be applied at a rate from about 1 to about 5,000 ppm, more preferably from about 10 to about 1,000 ppm, even more preferably from about 50 to about 1,000 ppm and even more preferably at about 70, about 270 or about 740 ppm.

In another embodiment, the present invention is directed to methods of improving plant part quality in plant parts subjected to post-harvest chilling temperatures comprising applying an effective amount of a mixture of S-ABA and a jasmonate to the plant part.

The methods of the present invention may exclude the application of nicotinamide.

The methods of the present invention may consist of applying an effective amount of a mixture of S-ABA and a jasmonate as the only agriculturally active ingredients applied.

In a preferred embodiment, the plant parts that may benefit from methods of the present invention includes, but are not limited to, fruits, vegetables, flowers and transplants.

Fruits that may benefit from methods of the present invention, include but are not limited to, avocados, pomegranates, bananas, mangoes, pineapples, eggplants, tomatoes, peppers, blueberries, raspberries, strawberries, blackberries, pome fruits such as apples and pears including European pears and Asian pears, grapes, kiwifruit, watermelon, and citrus such as oranges, grapefruit, mandarins, tangerines, lemons, and limes, and cultivars, varieties, and hybrids thereof.

Vegetables that may benefit from methods of the present invention, include but are not limited to, peas, carrots, sweet corn, sweet potatoes, potatoes, cucumber, lettuce, cabbage, broccoli, cauliflower, carrots, sweet basil, spinach, and beets and cultivars, varieties, and hybrids thereof.

Flowers that may benefit from methods of the present invention, include but are not limited to, carnations, roses, and tulips and cultivars, varieties, and hybrids thereof.

Cultivars, varieties and hybrids of plant parts may be from a plant which can be produced by natural hybridization, a plant which can occur as the result of a mutation, an F1 hybrid plant, or a transgenic plant (also referred to as a “genetically modified plant”).

The term “F1 hybrid plant” refers to a plant of a first filial generation which is produced by hybridizing two different varieties with each other, and is generally a plant which has a more superior trait to that of either one of parents thereof. The term “transgenic plant” refers to a plant which is produced by introducing a foreign gene from another organism such as a microorganism into a plant and which has a property that cannot be acquired easily by hybridization breeding, induction of a mutation or a naturally occurring recombination under a natural environment.

Examples of the technique for producing the above-mentioned plants include a conventional breeding technique, a transgenic technique, a genome-based breeding technique, a new breeding technique, and a genome editing technique. The conventional breeding technique is a technique for producing a plant having a desirable property by mutation or hybridization. The transgenic technique is a technique for imparting a new property to a specific organism by isolating a gene (DNA) of interest from the organism and then introducing the gene (DNA) into the genome of another target organism, and an antisense technique or an RNA interference technique which is a technique for imparting a new or improved property to a plant by silencing another gene occurring in the plant.

The genome-based breeding technique is a technique for increasing the efficiency of breeding using genomic information, and includes a DNA marker (also referred to as “genome marker” or “gene marker”) breeding technique and genomic selection. For example, the DNA marker breeding is a method in which an offspring having a desired useful trait gene is selected from many hybrid offspring using a DNA marker that is a DNA sequence capable of serving as an indicator of the position of a specific useful trait gene on a genome. The analysis of a hybrid offspring of a plant at a seedling stage thereof using the DNA marker has such a characteristic that it becomes possible to shorten the time required for breeding effectively.

The genomic selection is such a technique that a prediction equation is produced from a phenotype and genomic information both obtained in advance and then a property is predicted from the prediction equation and the genomic information without carrying out the evaluation of the phenotype. The genomic selection can contribute to the increase in efficiency of breeding. A “new breeding technique” is a collective term for a variety of breeding techniques including molecular biological techniques. Examples of the new breeding technique include techniques such as cisgenesis/intragenesis, oligonucleotide-directed mutagenesis, RNA-dependent DNA methylation, genome editing, grafting to a genetically modified rootstock or scion, reverse breeding, agroinfiltration, and seed production technology (SPT). The genome editing technique is a technique that converts genetic information in a sequence-specific manner, and enables addition, deletion and or substitution of a DNA base-pair sequence, addition, deletion and or substitution of an amino acid sequence, introduction of a foreign DNA base-pair sequence including genes and regulatory regions, and the like. Examples of the tool for the technique include zinc-finger nuclease (ZFN), TALEN, CRISPR/Cas9, CRISPER/Cpf1 and meganuclease which can cleave DNA in a sequence-specific manner, and a sequence-specific genome modification technique using CAS9 nickase, Target-AID and the like which is produced by any one of the modification of the above-mentioned tools. A skilled artisan would understand that future techniques will be developed that are capable of editing the genomic sequence, modifying transcription of a DNA sequence to an RNA sequence, modifying an RNA sequence, modifying translation of an RNA sequence to an amino acid sequence, modifying an amino acid sequence and or modifying the folding of an amino acid sequence and or agglomeration of amino acid sequences to a protein and that any or all of these techniques may be beneficial in modifying the phenotype of a plant. Plants whose phenotypes have been modified by all known and future techniques capable of modifying the phenotype of a plant are envisaged herein. Examples of the above-mentioned plant parts include parts of plants listed in genetically modified crops registration database (GM APPROVAL DATABASE) in an electric information site in INTERNATIONAL SERVICE for the ACQUISITION of AGRI-BIOTECH APPLICATIONS, ISAAA) (http://www.isaaa.org/).

In a preferred embodiment, the mixtures of the present invention may be disposed in a composition comprising one or more excipients selected from the group consisting of solvents, anti-caking agents, stabilizers, defoamers, slip agents, humectants, dispersants, wetting agents, thickening agents, emulsifiers, penetrants, adjuvants, synergists, polymers, propellants and preservatives.

The mixtures or compositions of the present invention can be applied by any convenient means. Those skilled in the art are familiar with the modes of application including but not limited to, spraying, brushing, soaking, dipping, drenching, granule application, pressurized liquids (aerosols), vapor and fogging. Spraying includes space sprays. Space sprays include aerosols and thermal fog spray. The mixtures and composition may further be mixed with a wax and/or edible coating and applied to the plant part.

The mixtures of the present composition may be applied concurrently or sequentially.

As used herein, the phrase “chilling temperatures” refers to those temperatures below 25 degrees Celsius that cause the quality of the plant part to diminish.

As used herein, the phrase “plant part quality” refers to any feature of the plant part that is desirable by the consumer including, but not limited to, texture, color, firmness, juiciness, aroma, and flavor. “Plant part quality” may also refer to scores on the hedonic scale, which are scores given by consumer taste panel assessments.

As used herein, “effective amount” refers to the amount of the S-ABA and/or a jasmonate that will improve cold stress tolerance, and/or plant part quality. The “effective amount” will vary depending on the S-ABA and the jasmonate concentrations, the plant species, variety, cultivar or hybrid being treated, the severity of the stress, the result desired, and the life stage of the plants, among other factors. Thus, it is not always possible to specify an exact “effective amount.” However, an appropriate “effective amount” in any individual case may be determined by one of ordinary skill in the art.

As used herein, “improving” means that the plant has more of the quality than the plant would have had it if it had not been treated by methods of the present invention.

As used herein, all numerical values relating to amounts, weight percentages and the like are defined as “about” or “approximately” each particular value, namely, plus or minus 10% (+10%). For example, the phrase “at least 5% by weight” is to be understood as “at least 4.5% to 5.5% by weight.” Therefore, amounts within 10% of the claimed values are encompassed by the scope of the claims.

The articles “a,” “an” and “the” are intended to include the plural as well as the singular, unless the context clearly indicates otherwise.

The disclosed embodiments are simply exemplary embodiments of the inventive concepts disclosed herein and should not be considered as limiting, unless the claims expressly state otherwise.

The following examples are intended to illustrate the present invention and to teach one of ordinary skill in the art how to use the formulations of the invention. They are not intended to be limiting in any way.

### EXAMPLES

### Example 1—Improved Stone Fruit Quality

### Method

Flamin' Fury® variety Peaches were harvested and maintained at room temperature until treatment. Peaches were divided into 9 equal sets. Each treatment type contained 15 peaches and was repeated 3 times for a total of 45 peaches per treatment. For treatment, water, (S)-abscisic acid (“S-ABA”), methyl jasmonate or a mixture of S-ABA and methyl jasmonate were applied to peaches after harvest via a 1-minute dip at the following concentrations: 1) water only; 2) 0.33 millimolar (“mM”) S-ABA; 3) 3.3 mM S-ABA; 4) 0.33 mM methyl jasmonate; 5) 3.3 mM methyl jasmonate; 6) 0.33 mM S-ABA and 0.33 mM methyl jasmonate; 7) 0.3 mM S-ABA and 3.3 mM methyl jasmonate; 8) 3.3 mM S-ABA and 0.33 mM methyl jasmonate or 9) 3.3 mM S-ABA and 3.3 mM methyl jasmonate. Each treatment includes 0.025% Latron B-1956® (available from J. R. Simplot Company) as an adjuvant. All peaches were subsequently dried for 30 minutes and then stored at 5 degrees Celsius for 4 weeks. Following cold storage all peaches were placed at room temperature for 1 day and then evaluated for flesh browning.

Flesh browning was determined using a 6-point scale developed by Crisosto G. M. 2015 wherein a score of 1=no browning, 2=very slight browning in the pit cavity, 3=slight browning in the pit cavity and surrounding tissue, 4=moderate browning on less than 50% of the flesh, 5=severe browning on 50% to 75% of the flesh and 6=extreme browning covering most of the flesh. A flesh browning score of more than 3 was determined as a “flesh browning incident” and a score of less than 3 was determined as “healthy fruit” for efficacy purposes.

To determine the percent of peaches that did not develop an undesirable amount of flesh browning (i.e. “healthy fruit”) the total number of peaches with a flesh browning score of less than 3 was divided by the total number of peaches per set.

To determine if the mixtures provided unexpected results, the observed combined efficacy (“OCE”) was divided by the expected combined efficacy (“ECE”) wherein the expected ECE is calculated by the Abbott method:

$$ECE = A + B - ( AB ) ,$$

wherein ECE is the expected combined efficacy and in which A and B are the quality score provided by application of the single active ingredients. If the ratio between the OCE of the mixture and the ECE of the mixture is greater than 1, then greater than expected interactions are present in the mixture. (Gisi, Synergistic Interaction of Fungicides in Mixtures, The American Phytopathological Society, 86:11, 1273-1279,1996). Results can be found in Table 1, below.

TABLE 1

| | Active Ingredient | | % Healthy | Fold Change | OCE:ECE |
|---|---|---|---|---|---|
| | Concentration | | Fruit | from Control | Ratio |
| Control | — | | 12.8 | — | — |
| S-ABA | 0.33 | mM | 17.3 | 0.35 | — |
| S-ABA | 3.3 | mM | 8.6 | −0.33 | — |
| MJ | 0.33 | mM | 28.0 | 1.18 | — |
| MJ | 3.3 | mM | 41.8 | 2.26 | — |
| S-ABA | 0.33 | mM | 34.1 | 1.66 | 1.5 |
| MJ | 0.33 | mM | | | |
| S-ABA | 0.33 | mM | 73.5 | 4.73 | 2.6 |
| MJ | 3.3 | mM | | | |
| S-ABA | 3.3 | mM | 43.9 | 2.43 | 1.9 |
| MJ | 0.33 | mM | | | |
| S-ABA | 3.3 | mM | 49.2 | 2.84 | 1.1 |
| MJ | 3.3 | mM | | | |

### Results

As seen in Table 1, above, application of a mixture of S-ABA and methyl jasmonate improved stone fruit quality in a greater than expected amount as compared to the addition of the improved quality provided by application of each alone at each of a 10:1, 1:1 and 1:10 concentration ratio.

### Example 2—Improved Stone Fruit Quality

### Method

Summer Flame 34 variety Peaches were harvested and maintained at room temperature until treatment. Peaches were divided into 4 equal sets. Each treatment type contained 32 peaches and was repeated 3 times for a total of 96 peaches per treatment. For treatment, water, (S)-abscisic acid (“S-ABA”), methyl jasmonate or a mixture of S-ABA and methyl jasmonate were mixed in Prima Fresh 55EU wax and applied in a semicommercial mini packing line to simulate commercial packing conditions. Active ingredients were mixed into the wax at the following concentrations: 1) wax only; 2) 1.2 mM S-ABA; 3) 1.2 mM methyl jasmonate; 4) 1.2 mM S-ABA and 1.2 mM methyl jasmonate. All peaches were immediately stored at 5 degrees Celsius for 25 days. Following cold storage all peaches were placed at room temperature for 3 day and then evaluated for flesh browning as described in Example 1, above and also for flesh mealiness.

Flesh mealiness was determined using a 3-point scale developed by Crisosto G. M. 2015 wherein a score of 1=juicy fruit, 2=moderately mealy fruit (small amount of juice released upon squeezing) and 3=severely mealy fruit (almost no juice released upon squeezing). A flesh mealiness score of 2 or more was determined as a “flesh mealiness incident” and a score of 1 was determined as “healthy fruit” for efficacy purposes.

Determination of unexpected results was calculated using the method described in Example 1, above. Results can be found in Table 2 for flesh browning and in Table 3 for flesh mealiness, below.

TABLE 2

| | Active Ingredient | Flesh Browning | Fold Change | OCE:ECE |
|---|---|---|---|---|
| | Concentration | Average Score | from Control | Ratio |
| Control | — | 3.08 | — | — |
| S-ABA | 1.2 mM | 3.03 | −0.016 | — |
| MJ | 1.2 mM | 2.79 | −0.09 | — |
| S-ABA | 1.2 mM | 2.58 | −0.16 | 1.5 |
| MJ | 1.2 mM | | | |

TABLE 3

| | Active | | | |
|---|---|---|---|---|
| Flesh | Ingredient | Flesh Mealiness | Fold Change | OCE:ECE |
| Mealiness | Concentration | Average Score | from Control | Ratio |
| Control | — | 1.67 | — | — |
| S-ABA | 1.2 mM | 1.66 | −0.006 | — |
| MJ | 1.2 mM | 1.50 | −0.10 | — |
| S-ABA | 1.2 mM | 1.39 | −0.17 | 1.5 |
| MJ | 1.2 mM | | | |

### Results

As seen in Tables 2 and 3, above, application of a mixture of S-ABA and methyl jasmonate improved stone fruit quality in a greater than expected amount as compared to the addition of the improved quality provided by application of each alone at a 1:1 and 1:10 concentration ratio.

### Example 3—Improved Cucumber Quality

### Method

Cucumbers were harvested and waxed and maintained at room temperature until treatment. Cucumbers were divided into 9 equal sets. Each treatment type contained 11-13 cucumbers peaches and was repeated 3 times for a total of 33-39 peaches per treatment. For treatment, water, (S)-abscisic acid (“S-ABA”), methyl jasmonate or a mixture of S-ABA and methyl jasmonate were applied to cucumber after harvest via a 2-minute dip at the following concentrations: 1) water only; 2) 0.3 millimolar (“mM”) S-ABA; 3) 3 mM S-ABA; 4) 0.3 mM methyl jasmonate; 5) 3 mM methyl jasmonate; 6) 0.3 mM S-ABA and 0.3 mM methyl jasmonate; 7) 0.3 mM S-ABA and 3 mM methyl jasmonate; 8) 3 mM S-ABA and 0.3 mM methyl jasmonate or 9) 3 mM S-ABA and 3 mM methyl jasmonate. Each treatment includes 0.025% Latron B-1956® (available from J. R. Simplot Company) as an adjuvant. All cucumbers were subsequently dried for 30 minutes and then stored at 5 degrees Celsius for 11.5 days. Following cold storage all cucumbers were placed at room temperature for 4 days and then evaluated for electrolyte leakage and decay on each of days 1, 2,3 and 4.

For electrolyte leakage cucumbers were sliced and a 1-centimeter core sample (i.e. a disc) was taken from each slice. 10 discs were placed in each 50-milliliter centrifuge tube containing 30 milliliter of deionized water and shaken for 25 minutes. Electroconductivity was measured at time zero as soon as the treatments were processed (CO). Electroconductivity was measured again after the discs were shaken in the centrifuge tubes (C1). Finally, electroconductivity was measured after boiling the cucumber sample for 20 minutes and then cooled rapidly (C2). % electrolyte leakage was calculated by the following formula (C1−C0)/C2.

Decay evaluation was performed by counting the number of cucumbers displaying decay and calculating the percentage of affected cucumbers. Decay severity was evaluated using a 1-3 score system, wherein 1=low, 2=medium and 3=high. Healthy fruit was calculated by subtracting the % of disease incidence from 100%.

Determination of unexpected results was calculated using the method described in Example 1. above. Results can be found in Table 4 for electrolyte leakage and in Table 5 for decay, below.

TABLE 4

| | Active Ingredient | | % Electrolyte | Fold Change | OCE:ECE |
|---|---|---|---|---|---|
| | Concentration | | Leakage | from Control | Ratio |
| Control | — | | 64.2 | — | — |
| S-ABA | 0.3 | mM | 61.3 | −0.05 | — |
| S-ABA | 3 | mM | 61.3 | −0.05 | — |
| MJ | 0.3 | mM | 60.3 | −0.06 | — |
| MJ | 3 | mM | 59.7 | −0.07 | — |
| S-ABA | 0.3 | mM | 42.6 | −0.34 | 3.1 |
| MJ | 0.3 | mM | | | |
| S-ABA | 0.3 | mM | 48.9 | −0.24 | 2.0 |
| MJ | 3 | mM | | | |
| S-ABA | 3 | mM | 37.5 | −0.42 | 3.8 |
| MJ | 0.3 | mM | | | |
| S-ABA | 3 | mM | 61.5 | −0.04 | 0.4 |
| MJ | 3 | mM | | | |

TABLE 5

| | Active Ingredient | | % Healthy | Fold Change | OCE:ECE |
|---|---|---|---|---|---|
| | Concentration | | Fruit | from Control | Ratio |
| Control | — | | 78.4 | — | — |
| S-ABA | 0.3 | mM | 82.1 | 0.05 | — |
| S-ABA | 3 | mM | 55.7 | −0.29 | — |
| MJ | 0.3 | mM | 61.5 | −0.22 | — |
| MJ | 3 | mM | 71.4 | −0.09 | — |
| S-ABA | 0.3 | mM | 56.6 | −0.28 | 1.8 |
| MJ | 0.3 | mM | | | |
| S-ABA | 0.3 | mM | 84.6 | 0.08 | −2.1 |
| MJ | 3 | mM | | | |
| S-ABA | 3 | mM | 47.0 | −0.40 | 0.7 |
| MJ | 0.3 | mM | | | |
| S-ABA | 3 | mM | 64.1 | −0.18 | 0.5 |
| MJ | 3 | mM | | | |

### Results

As seen in Table 4, above, application of a mixture of S-ABA and methyl jasmonate improved cucumber quality in a greater than expected amount as compared to the addition of the improved quality provided by application of each alone at a 101:1, 1:1 and 1:10 concentration ratio.

### Example 4—Improved Mango Quality (Prophetic)

### Method

Mangoes will be harvested and divided into groups for treatment. For treatment, water, (S)-abscisic acid (“S-ABA”), methyl jasmonate or a mixture of S-ABA and methyl jasmonate will be applied to mangoes after harvest via a 1-2 minute dip at the following concentrations: 1) water only; 2) 0.3 millimolar (“mM”) S-ABA; 3) 3 mM S-ABA; 4) 0.3 mM methyl jasmonate; 5) 3 mM methyl jasmonate; 6) 0.3 mM S-ABA and 0.3 mM methyl jasmonate; 7) 0.3 mM S-ABA and 3 mM methyl jasmonate; 8) 3 mM S-ABA and 0.3 mM methyl jasmonate or 9) 3 mM S-ABA and 3 mM methyl jasmonate. Each treatment will include 0.025% Latron B-1956® (available from J.R. Simplot Company) as an adjuvant. All mangoes will be subsequently dried for 30 minutes and then stored at chilling temperatures for one to several weeks. Following cold storage all mangoes will be placed at room temperature for one to several days and then evaluated for chilling injury symptoms.

Visual chilling injury symptoms (lenticel darkening, skin pitting, scalding, uneven ripening) of each individual mango will be assessed before and after transfer from the putative chilling temperatures to ambient temperature using a rating scale in which 1=severe, >50% of the fruit's surface showing damage; 2=moderate, 25-50% chilling damage; 3=slight, up to 25% pitting and/or scalding; 4=trace (small pits), 2-5% of the total fruit surface damaged; 5=no visible symptoms of injury.

Fruit firmness will be assessed on each individual mango by gentle hand pressure using a rating scale in which 1=fully soft, 2=advanced softness, 3=first softening, 4=firm to the touch, 5=very firm to the touch.

The shriveling of each individual mango will be assessed using a visual rating scale in which 1-extremely shriveled, wrinkled and dry, not acceptable under normal conditions; 2=severe shriveling, definitely, objectionable; 3=moderate, shriveling evident, becoming objectionable; 4=slight, minor signs of shriveling, not objectionable; 5=none, field fresh, no signs of shriveling.

The decay of each individual mango will be assessed using a modified visual rating scale from Horsfall and Barratt (1945) where 1=from 76% to 100% decay, severe to extreme decay (the mango is either partially or completely rotten); 2=from 51% to 75% decay, moderate to severe decay; 3=from 26% to 50% decay, slight to moderate decay (spots with decay and some mycelium growth); 4=from 1% to 25% decay, probable decay (brownish/grayish sunken minor spots); 5=0%, no decay.

### Results

As seen in peaches, the application of a mixture of S-ABA and methyl jasmonate is expected to improve mango quality in a greater than expected amount as compared to the addition of the improved quality provided by application of each alone.

### Example 5—Improved Avocado Quality (Prophetic)

### Method

Avocados will be harvested and divided into groups for treatment. For treatment, water, (S)-abscisic acid (“S-ABA”), methyl jasmonate or a mixture of S-ABA and methyl jasmonate will be applied to avocados after harvest via a 1-2 minute dip at the following concentrations: 1) water only; 2) 0.3 millimolar (“mM”) S-ABA; 3) 3 mM S-ABA; 4) 0.3 mM methyl jasmonate; 5) 3 mM methyl jasmonate; 6) 0.3 mM S-ABA and 0.3 mM methyl jasmonate; 7) 0.3 mM S-ABA and 3 mM methyl jasmonate; 8) 3 mM S-ABA and 0.3 mM methyl jasmonate or 9) 3 mM S-ABA and 3 mM methyl jasmonate. Each treatment will include 0.025% Latron B-1956® (available from J. R. Simplot Company) as an adjuvant. All avocados will be subsequently dried for 30 minutes and then stored at chilling temperatures for one to several weeks. Following cold storage all avocados will be placed at room temperature for one to several days and then evaluated for chilling injury symptoms.

Visual chilling injury symptoms including fruit peel defects and fruit flesh defects of each individual avocado will be assessed before and after transfer from the putative chilling temperatures to ambient temperature using a rating scale. For fruit peel defects the scale will be a relative scale scored 0 to 3 wherein 0=no chilling injury, 1=up to 25% surface area showing chilling injury, 2=from 26% to 50% surface area chilling injury and 3=from 50% to 100% chilling injury. For fruit flesh defects the scale will be a relative scale scored 0 to 3 wherein 0=no occurrence of vascular browning, 1=slight vascular browning, 2=moderate vascular browning and 3=severe vascular browning. Vascular browning is browning of the longitudinal vascular strands.

### Results

As seen in peaches, the application of a mixture of S-ABA and methyl jasmonate is expected to improve avocado quality in a greater than expected amount as compared to the addition of the improved quality provided by application of each alone.

### Example 6—Improved Bell Pepper Quality (Prophetic)

### Method

Bell peppers will be harvested and divided into groups for treatment. For treatment, water, (S)-abscisic acid (“S-ABA”), methyl jasmonate or a mixture of S-ABA and methyl jasmonate will be applied to bell peppers after harvest via a 1-2 minute dip at the following concentrations: 1) water only; 2) 0.3 millimolar (“mM”) S-ABA; 3) 3 mM S-ABA; 4) 0.3 mM methyl jasmonate; 5) 3 mM methyl jasmonate; 6) 0.3 mM S-ABA and 0.3 mM methyl jasmonate; 7) 0.3 mM S-ABA and 3 mM methyl jasmonate; 8) 3 mM S-ABA and 0.3 mM methyl jasmonate or 9) 3 mM S-ABA and 3 mM methyl jasmonate. Each treatment will include 0.025% Latron B-1956® (available from J. R. Simplot Company) as an adjuvant. All bell peppers will be subsequently dried for 30 minutes and then stored at chilling temperatures for one to several weeks. Following cold storage all bell peppers will be placed at room temperature for one to several days and then evaluated for chilling injury symptoms.

Visual chilling injury symptoms including sunken pitting on the skin or calyx and decay incidence of each individual bell pepper will be assessed before and after transfer from the putative chilling temperatures to ambient temperature using a rating scale. For sunken pitting the scale will be a relative scale scored 0 to 3 wherein 0=no chilling injury, 1=up to 25% surface area showing chilling injury, 2=from 26% to 50% surface area chilling injury and 3=from 50% to 100% chilling injury. For decay incidence, the number of bell peppers which show visual evidence of decay will be divided by the total number of bell peppers for each treatment group.

### Results

As seen in peaches, the application of a mixture of S-ABA and methyl jasmonate is expected to improve bell pepper quality in a greater than expected amount as compared to the addition of the improved quality provided by application of each alone.

### Example 7—Improved Banana Quality (Prophetic)

### Method

Bananas will be harvested and divided into groups for treatment. For treatment, water, (S)-abscisic acid (“S-ABA”), methyl jasmonate or a mixture of S-ABA and methyl jasmonate will be applied to bananas after harvest via a 1-2 minute dip at the following concentrations: 1) water only; 2) 0.3 millimolar (“mM”) S-ABA; 3) 3 mM S-ABA; 4) 0.3 mM methyl jasmonate; 5) 3 mM methyl jasmonate; 6) 0.3 mM S-ABA and 0.3 mM methyl jasmonate; 7) 0.3 mM S-ABA and 3 mM methyl jasmonate; 8) 3 mM S-ABA and 0.3 mM methyl jasmonate or 9) 3 mM S-ABA and 3 mM methyl jasmonate. Each treatment will include 0.025% Latron B-1956® (available from J. R. Simplot Company) as an adjuvant. All bananas will be subsequently dried for 30 minutes and then stored at chilling temperatures for one to several weeks. Following cold storage all bananas will be placed at room temperature for one to several days and then evaluated for chilling injury symptoms.

Visual chilling injury symptoms including surface discoloration, dull or smokey color, sub epidermal tissues revealing dark-brown streaks, failure to ripen and flesh browning of each individual banana will be assessed before and after transfer from the putative chilling temperatures to ambient temperature using a rating scale. For peel browning the scale will be a relative scale scored 0 to 4 wherein 0=no browning, 1=up to 25% surface area browning, 2=from 26% to 50% surface area browning, 3=from 50% to 75% surface area browning and 4=from 75% to 100% surface area browning. The peel browning index will be calculated as Σ (browning scale×proportion of fruit at each scale).

### Results

As seen in peaches, the application of a mixture of S-ABA and methyl jasmonate is expected to improve banana quality in a greater than expected amount as compared to the addition of the improved quality provided by application of each alone.

### Example 8—Improved Tomato Quality (Prophetic)

### Method

Tomatoes will be harvested and divided into groups for treatment. For treatment, water, (S)-abscisic acid (“S-ABA”), methyl jasmonate or a mixture of S-ABA and methyl jasmonate will be applied to tomatoes after harvest via a 1-2 minute dip at the following concentrations: 1) water only; 2) 0.3 millimolar (“mM”) S-ABA; 3) 3 mM S-ABA; 4) 0.3 mM methyl jasmonate; 5) 3 mM methyl jasmonate; 6) 0.3 mM S-ABA and 0.3 mM methyl jasmonate; 7) 0.3 mM S-ABA and 3 mM methyl jasmonate; 8) 3 mM S-ABA and 0.3 mM methyl jasmonate or 9) 3 mM S-ABA and 3 mM methyl jasmonate. Each treatment will include 0.025% Latron B-1956® (available from J. R. Simplot Company) as an adjuvant. All tomatoes will be subsequently dried for 30 minutes and then stored at chilling temperatures for one to several weeks. Following cold storage all tomatoes will be placed at room temperature for one to several days and then evaluated for chilling injury symptoms.

Visual chilling injury symptoms including peel color, peel pitting and decay incidence of each individual tomato will be assessed before and after transfer from the putative chilling temperatures to ambient temperature using a rating scale. For peel color the color will be assessed using a spectrophotometer. For peel pitting the scale will be a relative scale scored 0 to 3 wherein 0=no chilling injury, 1=up to 25% surface area showing chilling injury, 2=from 26% to 50% surface area chilling injury and 3=from 50% to 100% chilling injury. For decay incidence, the number of tomatoes which show visual evidence of decay will be divided by the total number of tomatoes for each treatment group.

### Results

As seen in peaches, the application of a mixture of S-ABA and methyl jasmonate is expected to improve tomato quality in a greater than expected amount as compared to the addition of the improved quality provided by application of each alone.

### Example 8—Improved Pome Fruit Quality (Prophetic)

### Method

Apples and/or pears will be harvested and divided into groups for treatment. For treatment, water, (S)-abscisic acid (“S-ABA”), methyl jasmonate or a mixture of S-ABA and methyl jasmonate will be applied to apples and/or pears after harvest via a 1-2 minute dip at the following concentrations: 1) water only; 2) 0.3 millimolar (“mM”) S-ABA; 3) 3 mM S-ABA; 4) 0.3 mM methyl jasmonate; 5) 3 mM methyl jasmonate; 6) 0.3 mM S-ABA and 0.3 mM methyl jasmonate; 7) 0.3 mM S-ABA and 3 mM methyl jasmonate; 8) 3 mM S-ABA and 0.3 mM methyl jasmonate or 9) 3 mM S-ABA and 3 mM methyl jasmonate. Each treatment will include 0.025% Latron B-1956® (available from J.R. Simplot Company) as an adjuvant. All apples and/or pears will be subsequently dried for 30 minutes and then stored at chilling temperatures for one to several weeks. Following cold storage all apples and/or pears will be placed at room temperature for one to several days and then evaluated for chilling injury symptoms.

Visual chilling injury symptoms including superficial scald of each individual apple and/or pear will be assessed before and after transfer from the putative chilling temperatures to ambient temperature using a rating scale. Superficial scald is a physiological disorder caused by chilling injury of apple and pears. It is manifested as brown or black patches on the fruit skin caused by oxidation of certain compounds located in the fruit skin (i.e. alpha-farnesene, a volatile terpene) that results in injury to the cell membranes causing cell death in the outermost cell layers of the fruit. For scalding injury, the scale will be a relative scale based on surface area coverage and scored 0 to 4 wherein 0=no scald, 1=up to 25% surface area showing scald, 2=from 26% to 50% surface area scald, 3=from 51% to 75% surface area scald and 4=from 76% to 100% surface area scald.

### Results

As seen in peaches, the application of a mixture of S-ABA and methyl jasmonate is expected to improve pome fruit quality in a greater than expected amount as compared to the addition of the improved quality provided by application of each alone.

### Example 9—Improved Citrus Fruit Quality (Prophetic)

### Method

Citrus fruit will be harvested and divided into groups for treatment. For treatment, water, (S)-abscisic acid (“S-ABA”), methyl jasmonate or a mixture of S-ABA and methyl jasmonate will be applied to citrus fruit after harvest via a 1-2 minute dip at the following concentrations: 1) water only; 2) 0.3 millimolar (“mM”) S-ABA; 3) 3 mM S-ABA; 4) 0.3 mM methyl jasmonate; 5) 3 mM methyl jasmonate; 6) 0.3 mM S-ABA and 0.3 mM methyl jasmonate; 7) 0.3 mM S-ABA and 3 mM methyl jasmonate; 8) 3 mM S-ABA and 0.3 mM methyl jasmonate or 9) 3 mM S-ABA and 3 mM methyl jasmonate. Each treatment will include 0.025% Latron B-1956® (available from J. R. Simplot Company) as an adjuvant. All citrus fruit will be subsequently dried for 30 minutes and then stored at chilling temperatures for one to several weeks. Following cold storage all citrus fruit will be placed at room temperature for one to several days and then evaluated for chilling injury symptoms.

Visual chilling injury symptoms including fruit peel defects such as pitting and brown discoloration including leathery brown, sunken areas on the rind of each individual citrus fruit will be assessed before and after transfer from the putative chilling temperatures to ambient temperature using a rating scale. Fruit scale defect scores will be a relative scale based on damage severity and scored 0 to 3 wherein 0=no damage, 1=low damage (e.g. up to 10% damage of surface area), 2=moderate damage (e.g. from 11% to 50% damage of surface area), 3=severe damage (e.g. more than 50% damage of surface area).

### Results

As seen in peaches, the application of a mixture of S-ABA and methyl jasmonate is expected to improve citrus fruit quality in a greater than expected amount as compared to the addition of the improved quality provided by application of each alone.

What is claimed is:

1. A method of improving cold post-harvest stress tolerance in a plant part comprising applying an effective amount of a mixture of (S)-abscisic acid (S-ABA) and a jasmonate to the plant part.

2. The method of claim 1, wherein the jasmonate is selected from the group consisting of jasmonic acid, methyl jasmonate and prohydrojasmon.

3. The method of claim 2, wherein the jasmonate is methyl jasmonate.

4. The method of claim 1, wherein application occurs from about 4 weeks prior to harvest to about 2 weeks after harvest.

5. The method of claim 4, wherein application occurs after harvest.

6. The method of claim 1, wherein the plant part is subjected to chilling temperatures following application.

7. The method of claim 6, wherein the chilling temperatures are from about 0.5 to about 20 degrees Celsius.

8. The method of claim 6, wherein plant part quality is improved compared to plant parts that did not receive an application of the mixture of S-ABA and a jasmonate prior to being subjected to chilling temperatures.

9. The method of claim 1, wherein the mixture is applied at a weight ratio from about 100:1 to about 1:100 S-ABA to the jasmonate.

10. The method of claim 9, wherein the mixture is applied at a weight ratio from about 3:1 to about 1:3 S-ABA to the jasmonate.

11. The method of claim 1, wherein S-ABA is applied at a rate of from about 1 to about 5,000 parts per million.

12. The method of claim 11, wherein S-ABA is applied at a rate of from about 10 to about 1,000 parts per million.

13. The method of claim 1, wherein the jasmonate is applied at a rate of from about 1 to about 5,000 parts per million.

14. The method of claim 13, wherein the jasmonate is applied at a rate of from about 10 to about 1,000 parts per million.

15. The method of claim 1, wherein the plant part is selected from the group consisting of fruits, vegetables, and flowers.

16. The method of claim 15, wherein the plant part is a fruit selected from the group consisting of avocados, pomegranates, bananas, mangoes, pineapples, eggplants, tomatoes, peppers, blueberries, raspberries, strawberries, blackberries, pome fruits, grapes, kiwifruit, watermelon, and citrus such as oranges, grapefruit, mandarins, tangerines, lemons, and limes, and cultivars, varieties and hybrids thereof.

17. The method of claim 15, wherein the plant part is a vegetable selected from the group consisting of peas, carrots, sweet corn, sweet potatoes, potatoes, cucumber, lettuce, cabbage, broccoli, cauliflower, carrots, sweet basil, spinach, and beets and cultivars, varieties and hybrids thereof.

18. The method of claim 15, wherein the plant part is a flower selected from the group consisting of carnations, roses, and tulips and cultivars, varieties and hybrids thereof.

19. A method of improving plant part quality in plant parts subjected to post-harvest chilling temperatures comprising applying an effective amount of a mixture of (S)-abscisic acid (S-ABA) and a jasmonate to the plant part.

20. The method of claim 19, wherein application occurs prior to the plant part being subjected to chilling temperatures.

21. The method of claim 19, wherein chilling temperatures are from about 0.5 to about 20 degrees Celsius.

22. The method of claim 19, wherein plant part quality is improved compared to plant parts not receiving an application of the mixture of S-ABA and the jasmonate.
